(19) United States
(12) Patent Application Publication
(10) Pub. No.: US 2019/0270828 A1
(43) Pub. Date: Sep. 5, 2019

(54) TANDEM ANTIBODIES

(71) Applicant: ATGC INC., Wynnewood, PA (US)

(72) Inventor: XIAOFENG XIA, WYNNEWOOD, PA (US)

(73) Assignee: ATGC INC., Wynnewood, PA (US)

(21) Appl. No.: 16/291,958

(22) Filed: Mar. 4, 2019

Related U.S. Application Data
(60) Provisional application No. 62/638,428, filed on Mar. 5, 2018.

(51) Int. Cl. C07K 16/46; A61P 35/00; C07K 16/28

(52) U.S. Cl. CPC C07K 16/468; A61P 35/00; C07K 16/2803; C07K 2317/31; C07K 2317/55; C07K 2317/14; C07K 2317/622

(57) ABSTRACT

This document provides methods and materials related to self-masking tandem antibodies, for example, Self-Masking Anti-tumoR Tandem antibody (SMART-body). For instance, a self-masking tandem antibody may contain two antibody-based domains (each of which contains an antigen-binding site) that are connected by a linker peptide cleavable by a protease, and the connection between the two antibody-based domains results in the masking of at least one of the antigen-binding sites. In some embodiments, for example, the SMART-body is first provided as a single molecule which does not bind antigens because of the masking of the antigen-binding sites within the molecule, but subsequently, the linker peptide is cleaved by a protease expressed in a tumor microenvironment, severing the connection between and removing the masking of the two antibody-based domains, which can then bind antigens expressed in the tumor, achieving localized, targeted cancer therapy.

### CROSS REFERENCE TO RELATED APPLICATIONS

This application claims the benefits of U.S. Provisional App. No. 62/638,428, Attorney Docket No. ATGC-001-1P, filed on Mar. 5, 2018, the entire contents of which are hereby incorporated by reference.

### FIELD OF PRESENT DISCLOSURE

This present disclosure relates to self-masking tandem antibodies, for example, Self-Masking Anti-tumoR Tandem antibody (SMART-body).

### BACKGROUND INFORMATION

An antibody recognizes an antigen through a paratope it contains that specifically interacts with a particular epitope on the antigen it binds. Such specific antibody-antigen interactions have wide applications, including applications in cancer therapies. Various cancer therapies use antibodies to recognize tumor specific biomarkers, which can then trigger molecular and cellular mechanisms to kill the cancer cells, or kill the cancer cells by cytotoxic molecules conjugated to the antibodies. Dozens of antibody-based molecules have been approved by regulatory authorities in major markets, and hundreds of antibody-based molecules are in Phase I to Phase III clinical trials for various applications, including applications in cancer therapies.

An antibody that recognizes only one antigen, when used in cancer therapies, can lose its effect when cancer cells lose the antigen following natural or therapy-induced relapse.

An antibody that is constitutively active in binding an antigen, when used in cancer therapies, can cause off-tumor toxicities, resulting in severe side effects, because the antigen may also be expressed in off-tumor environments. And targeting multiple antigens (or biomarkers, as they are called sometimes) with multiple antibodies is more likely to cause severe off-tumor toxicity that can be harmful to the patients.

### SUMMARY

In one aspect, this document relates to a self-masking tandem antibody containing a first antibody-based domain containing a first antigen-binding site, and a second antibody-based domain containing a second antigen-binding site. The first antibody-based domain and the second antibody-based domain are connected by a linker peptide that is cleavable by a protease, and the connection between the first antibody-based domain and the second antibody-based domain results in the masking of at least one of the first antigen-binding site and the second antigen-binding site.

In another aspect, this document relates to a method for validating a design of a self-masking tandem antibody, including the steps of (a) testing the antigen binding of a first antibody-based domain containing a first antigen-binding site and a second antibody-based domain containing a second antigen-binding site, before the first antibody-based domain and the second antibody-based domain are connected by a linker peptide in a subsequent step, to make sure the first antigen-binding site by itself binds to a first antigen, and the second antigen-binding site by itself binds to a second antigen; (b) connecting the first antibody-based domain and the second antibody-based domain using a linker peptide to form a tandem antibody, and thereafter testing the tandem antibody for antigen binding to the first antigen and the second antigen; (c) if the tandem antibody still binds the first antigen or the second antigen, re-design the antibody-based domains and the linker peptide before starting at step (a) to test the new design again; (d) if the tandem antibody no longer binds at least one of the first antigen and the second antigen, cleaves the linker peptide with a protease, and then test if the antibody-based domains bind the respective antigens; (e) if the antibody-based domains do not bind the respective antigens, re-design the antibody-based domains and the linker peptide before starting at step (a) to test the new design again; and (f) if the antibody-based domains bind the respective antigens, then the design of the tandem antibody is validated.

In some implementations, a pharmaceutical composition containing a self-masking tandem antibody validated by the method above may be provided, and such a validated tandem antibody may be provided for use in cancer therapy.

In another aspect, a method for the preparation of a self-masking tandem antibody may be provided, containing (a) transforming a host cell with vectors comprising nucleic acid molecules encoding a first antibody-based domain containing a first antigen-binding site, and a second antibody-based domain containing a second antigen-binding site, wherein the first antibody-based domain and the second antibody-based domain are connected by a linker peptide that is cleavable by a protease, and the connection between the first antibody-based domain and the second antibody-based domain results in the masking of at least one of the first antigen-binding site and the second antigen-binding site; (b) culturing the host cell under conditions that allow synthesis of the tandem antibody; and (c) recovering the tandem antibody from the culture.

Implementations may include one or more of the following features. The first and second antigen-binding sites may bind antigens expressed on cancer. The protease may be expressed in a tumor microenvironment. The protease may be a matrix metalloproteinase (MMP), matriptase, urokinase, or cathepsin B. One or more blocking peptides may be connected to the linker peptide and contributing to the masking of one or more antigen-binding sites. The antibody-based domain may be a single-domain antibody (sdAb), a single-chain variable fragment (scFv), an antigen-binding fragment (Fab or F(ab′)2), or a full antibody (full Ab). The antibody-based domain may be connected with an effector molecule.

These general and specific aspects may be implemented using a system, a method, or a computer program, or any combinations of systems, methods, and computer programs. Other aspects, features, and advantages will be apparent from the following detailed description, the drawings, and the claims.

### BRIEF DESCRIPTION OF THE DRAWINGS

In the drawings:

FIG. 1 is a schematic diagram showing the working mechanism of a self-masking tandem antibody;

FIG. 2 is a schematic diagram showing the structure of a self-masking tandem antibody;

FIG. 3 is a schematic diagram showing how the antigen-binding sites on a self-masking tandem antibody can be masked;

FIG. 4A-FIG. 4D is a schematic diagram showing the antibody-based domains of a self-masking tandem antibody can be made in various formats;

FIG. 5 is a schematic diagram showing a self-masking tandem antibody can further be attached with one or more effector molecules; and

FIG. 6 is a schematic diagram showing a method for validating a design of a self-masking tandem antibody.

### DETAILED DESCRIPTION

This document provides methods and materials related to self-masking tandem antibodies. A tandem antibody as disclosed by this document, when used in cancer therapy, may be termed a Self-Masking Anti-tumoR Tandem antibody (SMART-body). The self-masking tandem antibodies described here can include two or more antibody-based domains (each of which contains an antigen-binding site) that are connected by a linker peptide that can be cleaved by a protease; the connections between or among the antibody-based domains can result in the masking of the antigen-binding sites, rendering the antibody-based domains inactive in binding the respective antigens, until after they are separated when the linker peptide connecting them is cleaved by a protease.

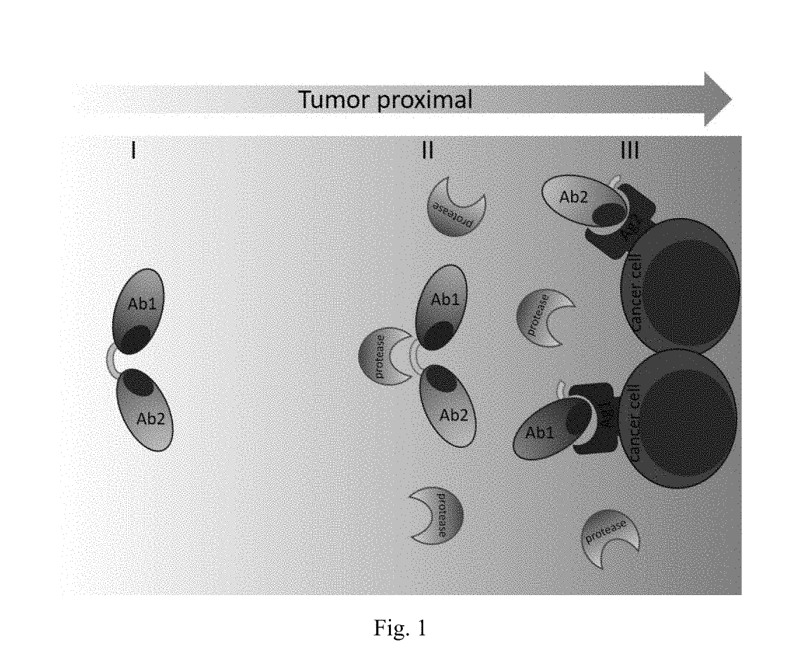

FIG. 1 illustrates the working mechanism of a SMART-body: (I) SMART-body is inactive at first because the antigen binding site in each antibody-based domain is masked by other domain(s) within the molecule; (II) in the tumor microenvironment, the linker peptide is recognized by a tumor-specific protease, and SMART-body is cleaved to generate multiple molecules, each with antigen-binding activities; and (III) the now disconnected, separate antibody-based molecules bind to their respective antigens (i.e., targets) and function to kill tumor cells. Prior to being cleaved by a protease, a self-masking tandem antibody can be inactive in antigen-binding in healthy blood or tissues. The linker peptide can be designed such that it can be cleaved by the proteases overexpressed in tumor cells and/or stromal compartment. Many such proteases are known and new ones may be discovered, examples of which include, but are not limited to, Matrix metalloproteinases (MMPs, e.g. MMP2, MMP9, MMP14), matriptase, μPA, and cathepsin B, among others. When a SMART-body approaches the tumor, the linker peptide is cleaved in the tumor microenvironment (FIG. 1, II) so that the SMART-body turns into multiple antibody-based molecules that can bind targets separately (FIG. 1, III). The binding will trigger further molecular or cellular mechanisms to kill the tumor cells. The targets for a SMART-body can be on the tumor cells, or on a non-tumor effector cell (e.g., the PD-1 protein on T cells). The targets may also be secreted proteins.

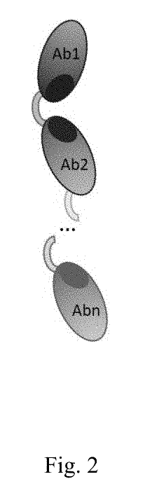

FIG. 2 illustrate the structure of a self-masking tandem antibody (including a SMART-body). The self-masking tandem antibody can contain a tandem of two (or more) antibody-based molecules connected to each other (or one another) by a linker peptide that can be cleaved by a protease, such as a tumor-specific protease. As a result of the connection, some (at least one) or all antigen-binding sites are masked, preventing their antigen-binding activities. The linker peptides used in a self-masking tandem antibody (including a SMART-body) can be the same or different.

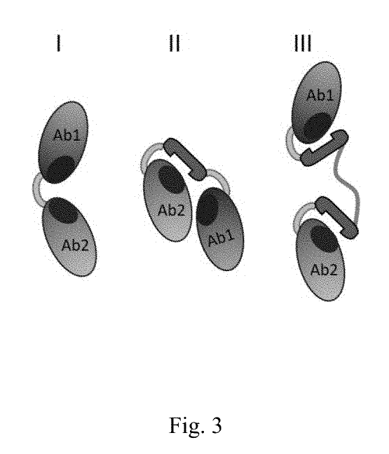

FIG. 3 shows how the antigen-binding sites on a self-masking tandem antibody can be masked. A self-masking tandem antibody may or may not contain an additional blocking peptide (phone-shaped) to achieve masking. The masking can be achieved through the steric hindering from another antibody-based domain (or domains) without a blocking peptide (FIG. 3 I). The masking of some or all of the antigen-binding sites within a self-masking tandem antibody can also be achieved using blocking peptide attached to some (FIG. 3 II) or all (FIG. 3 III) of the antibody-based domains.

A self-masking tandem antibody (including a SMART-body) can be made by either recombinant protein expression or chemical conjugation. By selecting appropriate constituent antibodies, and optimizing the connection site (e.g., choosing C or N terminal fusion through recombinant protein expression, or amino acid specific conjugation chemically), SMART-body can be constructed so that at least one antigen-binding site is masked though the steric hindering by another antibody-based domain (or domains) without the need for a blocking peptide (FIG. 3 I).

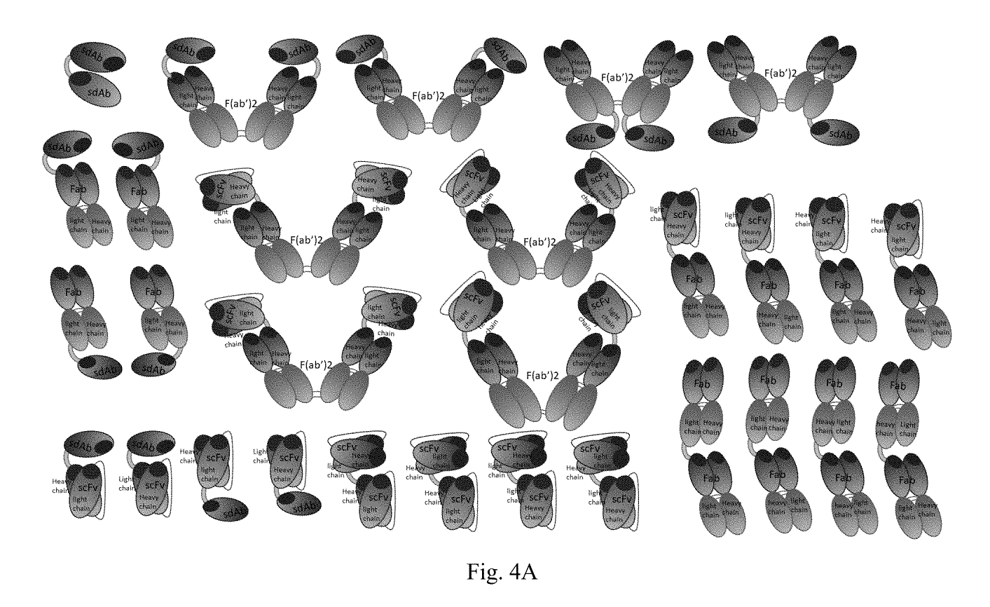

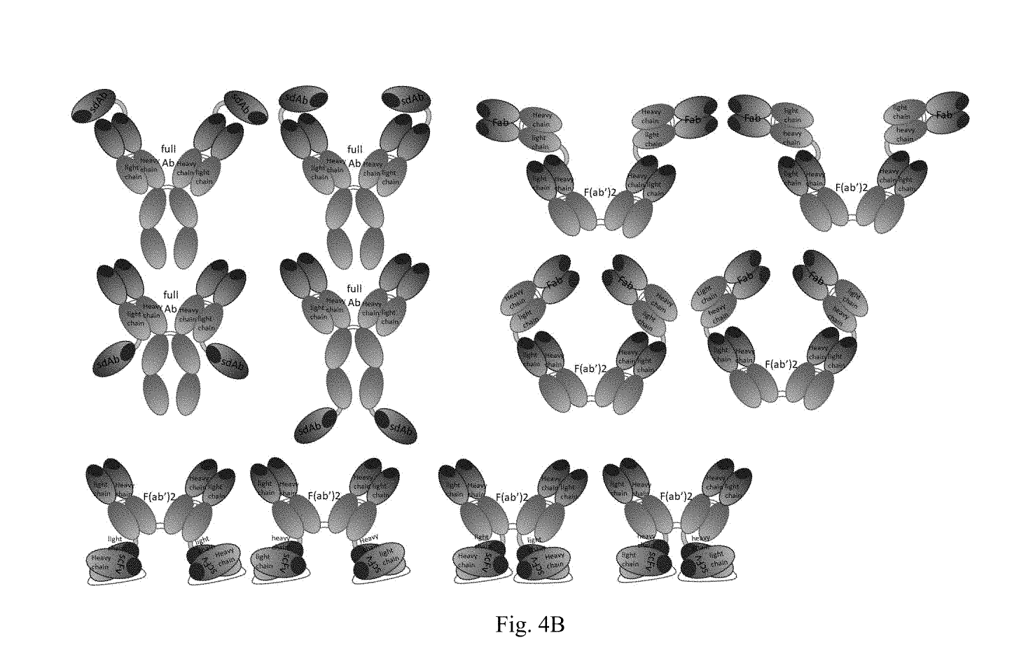

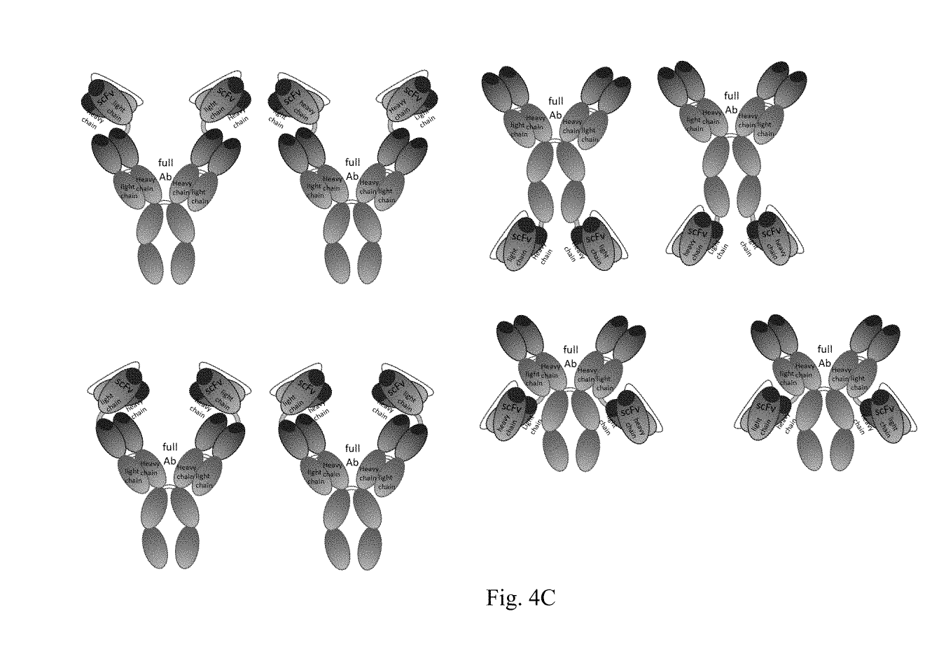

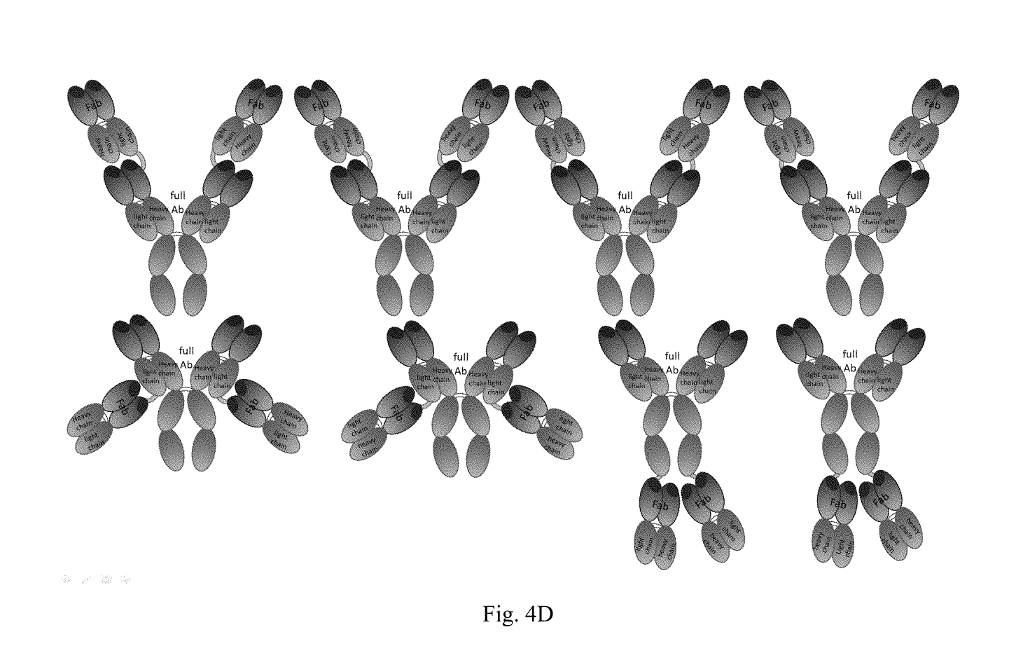

FIG. 4A-FIG. 4D shows that the antibody-based domains of a self-masking tandem antibody can be made in various formats. In some implementations, single chain antibodies are used, such as the single domain antibodies (sdAb, including the camelid nanobody (VHH) and shark V-NAR), and the single-chain variable fragment (scFv), which can be fused together through recombinant protein expression. Other formats, such as antigen-binding fragment (Fab), F(ab′)₂ and full antibody (full Ab), can also be used. When antibodies comprising both heavy chains and light chains are used, the antibody-based domains can be connected with either the heavy chains or light chains, as illustrated in FIG. 4A-FIG. 4D.

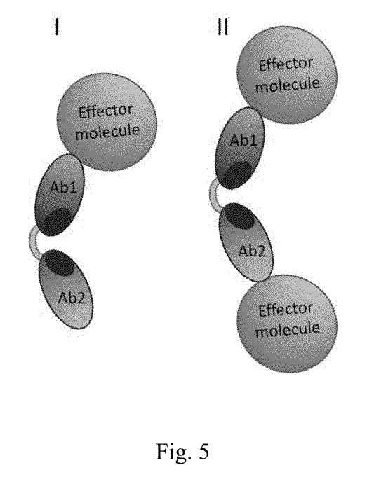

FIG. 5 shows a self-masking tandem antibody can further be attached with one or more effector molecules. In some implementations, to improve its therapeutic function, SMART-body can be attached with an effector molecule either by recombinant protein expression or chemical conjugation. The effector molecule(s) can be attached to some (FIG. 5, I) or all of the antibody domains (FIG. 5, II). The effector molecule can be proteins (such as Fc domain, human serum albumin (HSA), anti-HAS) to increase the half-life in blood. The effector molecule can also be cytotoxic drugs such as the molecules used in antibody-drug conjugates (ADCs) (e.g., maytansinoid DM1, MMAE, calicheamicin cytotoxin, etc.). The effector molecule can also be used for the purpose of increasing steric hindrance to achieve antibody affinity masking.

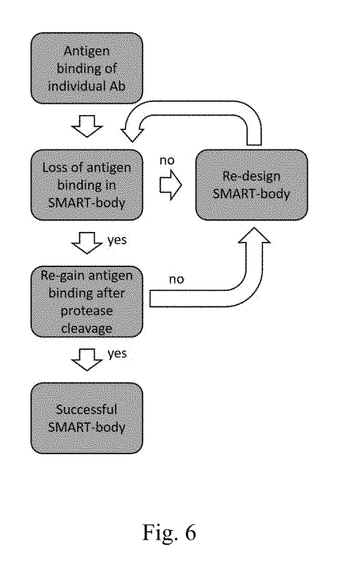

FIG. 6 is a schematic flow chart depicting a method for validating a design of a self-masking tandem antibody. In some implementations, a self-masking tandem antibody, including a SMART-body, can be screened and tested following the procedures exemplified in FIG. 6. A panel of individual constituent antibodies in various formats (as illustrated in FIG. 4A-FIG. 4D) can be selected to prove their antigen-binding selectivity and affinity first. They are then engineered into a SMART-body by recombinant protein expression or chemical conjugation. The SMART-body will then be tested until a loss of antigen-binding construct is identified. The selected SMART-body will then be protease cleaved, and the product tested again for antigen-binding. The construct re-gaining the antigen-binding affinity and specificity is identified as a successful SMART-body lead.

In some implementations, a pharmaceutical or a diagnostic composition containing a self-masking tandem antibody validated by the method above may be provided, and such a validated self-masking tandem antibody may be provided for use in cancer therapy, or for diagnostic purposes.

In some implementations, a pharmaceutical composition containing a self-masking tandem antibody obtained as disclosed herein can be provided, with at least one pharmaceutically acceptable excipient.

In some implementations, a patient in need of cancer therapy may be treated by administering to the patient a therapeutically effective amount of a self-masking tandem antibody obtained as disclosed herein.

In some implementations, a method for the preparation of a self-masking tandem antibody may be provided, containing (a) transforming a host cell with vectors comprising nucleic acid molecules encoding a first antibody-based domain containing a first antigen-binding site, and a second antibody-based domain containing a second antigen-binding site, wherein the first antibody-based domain and the second antibody-based domain are connected by a linker peptide that is cleavable by a protease, and the connection between the first antibody-based domain and the second antibody-based domain results in the masking of at least one of the first antigen-binding site and the second antigen-binding site; (b) culturing the host cell under conditions that allow synthesis of the tandem antibody; and (c) recovering the self-masking tandem antibody from the culture.

The self-masking tandem antibodies as described herein may be produced by recombinant protein expression. Such production methods are widely known in the state of the art and include recombinant protein expression in both prokaryotic and eukaryotic cells with subsequent isolation of the antibody polypeptide and purification to a pharmaceutically acceptable purity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A self-masking tandem antibody, comprising: a) a first antibody-based domain containing a first antigen-binding site, and b) a second antibody-based domain containing a second antigen-binding site,
wherein the first antibody-based domain and the second antibody-based domain are connected by a linker peptide that is cleavable by a protease; and
wherein the connection between the first antibody-based domain and the second antibody-based domain results in the masking of at least one of the first antigen-binding site and the second antigen-binding site.

2. The self-masking tandem antibody of claim 1, wherein the first and second antigen-binding sites bind antigens expressed on cancer cells.

3. The self-masking tandem antibody of claim 1, wherein the protease is expressed in a tumor microenvironment.

4. The self-masking tandem antibody of claim 1, wherein the protease is a matrix metalloproteinase (MMP).

5. The self-masking tandem antibody of claim 1, wherein the protease is selected from the group consisting of matriptase, urokinase, and cathepsin B.

6. The self-masking tandem antibody of claim 1, further comprising one or more blocking peptides connected to the linker peptide and contributing to the masking of one or more antigen-binding sites.

7. The self-masking tandem antibody of claim 1, wherein the antibody-based domain is a single-domain antibody (sdAb).

8. The self-masking tandem antibody of claim 1, wherein the antibody-based domain is a single-chain variable fragment (scFv).

9. The self-masking tandem antibody of claim 1, wherein the antibody-based domain is an antigen-binding fragment (Fab or F(ab′)₂).

10. The self-masking tandem antibody of claim 1, wherein the antibody-based domain is a full antibody (full Ab).

11. The self-masking tandem antibody of claim 1, wherein the antibody-based domain is further connected with an effector molecule.

12. A method for validating a design of a self-masking tandem antibody, comprising:
a) testing the antigen binding of a first antibody-based domain containing a first antigen-binding site and a second antibody-based domain containing a second antigen-binding site, before the first antibody-based domain and the second antibody-based domain are connected by a linker peptide in a subsequent step, to make sure the first antigen-binding site by itself binds to a first antigen, and the second antigen-binding site by itself binds to a second antigen;
b) connecting the first antibody-based domain and the second antibody-based domain using a linker peptide to form a tandem antibody, and thereafter testing the tandem antibody for antigen binding to the first antigen and the second antigen;
c) if the tandem antibody still binds both the first antigen and the second antigen, re-design the antibody-based domains and the linker peptide before starting at step a) to test the new design again;
d) if the tandem antibody no longer binds at least one of the first antigen and the second antigen, cleaves the linker peptide with a protease, and then test if the antibody-based domains bind the respective antigens;
e) if the antibody-based domains do not bind the respective antigens, re-design the antibody-based domains and the linker peptide before starting at step a) to test the new design again; and
f) if the antibody-based domains bind the respective antigens, then the design of the tandem antibody is validated.

13. The method of claim 12, wherein the antibody-based domain is a single-domain antibody (sdAb).

14. The method of claim 12, wherein the antibody-based domain is a single-chain variable fragment (scFv).

15. The method of claim 12, wherein the protease is expressed in a tumor microenvironment.

16. The method of claim 12, wherein the protease is a matrix metalloproteinase (MMP).

17. The method of claim 12, wherein the protease is selected from the group consisting of matriptase, urokinase, and cathepsin B.

18. A pharmaceutical composition containing a self-masking tandem antibody validated by the method of claim 12.

19. A self-masking tandem antibody validated by the method of claim 12 for use in cancer therapy.

20. A method for the preparation of a self-masking tandem antibody, comprising:
a) transforming a host cell with vectors comprising nucleic acid molecules encoding a first antibody-based domain containing a first antigen-binding site, and a second antibody-based domain containing a second antigen-binding site,
wherein the first antibody-based domain and the second antibody-based domain are connected by a linker peptide that is cleavable by a protease; and
wherein the connection between the first antibody-based domain and the second antibody-based domain results in the masking of at least one of the first antigen-binding site and the second antigen-binding site.
b) culturing the host cell under conditions that allow synthesis of the tandem antibody; and
c) recovering the tandem antibody from the culture.
